SEQUENCE LISTING (6 sequences)

SEQ ID NO: 1 - DNA, 40 bp, Artificial Sequence
  Primer, FIP
ccggttgaatgctcttcccgggcaaagggagcttggtgac

SEQ ID NO: 2 - DNA, 40 bp, Artificial Sequence
  Primer, BIP
gctatcagtgcatggctcccaggcgtaacctcgactttcg

SEQ ID NO: 3 - DNA, 20 bp, Artificial Sequence
  Primer, LF
aacacgtaaacttggcacat

SEQ ID NO: 4 - DNA, 21 bp, Artificial Sequence
  Primer, LB
agcgggatgattggatatgaa

SEQ ID NO: 5 - DNA, 19 bp, Artificial Sequence
  Primer, F3
gggaaacggttgtggactt

SEQ ID NO: 6 - DNA, 19 bp, Artificial Sequence
  Primer, B3
gcttccgctcttggtgaat

(19) United States
(12) Patent Application Publication
(10) Pub. No.: US 2020/0063197 A1
(43) Pub. Date: Feb. 27, 2020

(54) QUANTITATIVE DETECTION AND ANALYSIS OF TARGET DNA WITH COLORIMETRIC RT-QLAMP

(71) Applicant: ARIZONA BOARD OF REGENTS ON BEHALF OF ARIZONA STATE UNIVERSITY, Scottsdale, AZ (US)

(72) Inventors: Deirdre Meldrum, Phoenix, AZ (US); Shufang Ci, Tempe, AZ (US); Shih-Hui (Joseph) Chao, Phoenix, AZ (US); Rhett Martineau, Gilbert, AZ (US); Weimin Gao, Chandler, AZ (US)

(21) Appl. No.: 16/611,799

(22) PCT Filed: May 15, 2018
(86) PCT No.: PCT/US2018/032706

§ 371 Date: Nov. 7, 2019

Related U.S. Application Data
(60) Provisional application No. 62/506,428, filed on May 15, 2017.

(51) Int. Cl. C12Q 1/6851; G16B 40/10; C12Q 1/70; G01N 21/78; G01N 21/27; G06T 7/90; G06T 7/00

(52) U.S. Cl. CPC C12Q 1/6851; G16B 40/10; C12Q 1/702; G01N 21/78; G06T 2207/30072; G06T 7/90; G06T 7/0012; G06T 2207/10024; G01N 21/272

(57) ABSTRACT

The present disclosure relates to real-time quantification using loop-mediated isothermal amplification, in particular real-time colorimetric reverse transcription quantitative loop-mediated isothermal amplification (RT-qLAMP). In some embodiments, RT-qLAMP is used to diagnose the presence of and also quantitate the amount of Zika virus in a sample.

### CROSS-REFERENCE TO RELATED APPLICATIONS

This application claims the benefit of U.S. Provisional Patent Application Ser. No. 62/506,428, filed on May 15, 2017, the contents of which are incorporated herein by reference.

### REFERENCE TO SEQUENCE LISTING SUBMITTED ELECTRONICALLY

The official copy of the sequence listing is submitted electronically via EFS-Web as an ASCII-formatted sequence listing with a file named “11157_017PCT_SeqList.txt” created on May 1, 2017 and having a size of 1,418 bytes and is filed concurrently with the specification. The sequence listing contained in this ASCII-formatted document is part of the specification and is herein incorporated by reference in its entirety.

### TECHNICAL FIELD

The disclosure relates to rapid diagnostic technologies, in particular, technologies related to loop-mediated isothermal application (LAMP). The disclosure also relates to real-time quantification using LAMP. Additionally, the disclosure relates to the detection and quantification of Zika virus in a sample.

### BACKGROUND

Loop-mediated isothermal amplification (LAMP) is a rapid and sensitive gene amplification method. LAMP reactions are conducted at a single temperature (usually between 60-65° C.), and results are usually obtained within one hour. In the past few years, LAMP has been integrated into lab-on-a-chip technologies.

The technique is based on a polymerase with strand displacement activity and three pairs of primers. LAMP is generally considered to be highly specific, because the three pairs of primers recognize six distinct regions in the target DNA. LAMP products—and thus the presence of an analytical target of interest in an unknown sample—can be assessed through a wide variety of detection chemistries. Turbidity measurement bioluminescence, fluorescence via intercalating dyes, and even fluorescence by removal of fluorophore quenchers are among the most commonly used real-time detection methods to date. Drawbacks to these methods include poor signal-to-noise ratio and high susceptibility to measurement artifacts (turbidity), increased rates of false positives (intercalating dyes) and high cost of reagents, detectors, or both (bioluminescence, fluorophore quenching, and intercalating dyes). Colorimetric reagents, including metal ion indicators such as hydroxyl naphthol blue (HNB) and pH indicators such as cresol red may offer the best performance to cost values. For both reagents, fast differentiation between positive and negative reactions has been reported. However, real-time quantitative results have not been achieved using either colorimetric method.

Effective, rapid detection methods are greatly needed for epidemics, for example, the Zika epidemic. In addition to diagnosis of a Zika virus (ZIKV) infection, there is also a need of a quantitative tool to measure the viral burden of ZIKV. The Zika crisis is growing and affecting the public health in the Americas. Six years after the first reported Zika epidemic in Yap State in 2007, there was a larger Zika outbreak in French Polynesia in 2013. Since then, Zika fever has been actively circulating in the Pacific islands. During 2015-2016, Zika fever arrived and was spreading rapidly in the Americas. As of Aug. 31, 2016, it has been actively transmitting in more than 50 countries and territories throughout the world. The ongoing Zika crisis is caused by Zika virus (ZIKV). ZIKV is a species from the flavivirus genus, and is usually transmitted by mosquitoes. It is an enveloped virus with approximately 10,676 bases single-stranded positive-sense RNA genome. ZIKV has been reported to be highly associated with a neurological disorder called Guillain-Barré syndrome in adults, and recently confirmed to cause Microcephaly in neonates.

Unfortunately, the development to detect this emerging infectious disease is in its infancy. Various groups have demonstrated the detection of ZIKV from a variety of clinical samples. However, since the clinical presentation of Zika fever is similar to those of chikungunya and dengue fever, Zika fever may be misclassified as other diseases, increasing the difficulty for ZIKV infection diagnosis. Currently, there are two main types of methods routinely used in laboratory conditions to diagnose ZIKV: RT-PCR based molecular tests and ELISA based serological tests. The FDA has published a list of Zika diagnostic kits under Emergency Use Authorizations (for example, LightMix® Zika rRT-PCR Test from Roche). Even though these technologies identified a large amount of Zika cases, false positives and cross-reactions with other flaviviruses (for example, dengue virus) occur frequently. RT-PCR based detection methods are also very limited in front-line performance for point-of-care diagnostics, which forms a bottleneck to assess the pandemic situation.

Song et al. (“Instrument-Free Point-of-Care Molecular Detection of Zika Virus.” Anal. Chem. 2016, 88, 7289-7294) reported a portable microfluidic cassette for qualitative field detection of Zika using the end point colorimetric RT-LAMP technology. The platform was reported with a high sensitivity for ZIKV detection. However, no real-time ZIKV RNA quantification has been achieved using colorimetric reverse transcription LAMP (RT-LAMP). With the ability to quantify the amount of ZIKV in a sample, researchers can quantitatively measure ZIKV RNA loads to evaluate pathogen reduction efficacy in the development of vaccines, medications, or treatments for ZIKV infections. An effective treatment should be able to reduce pathogen load by 6 to 10 log copies/mL in a blood product. In addition, for point-of-care applications, the diagnostics technology should be inexpensive and rapid. Pardee et al. (“Rapid, Low-Cost Detection of Zika Virus Using Programmable Biomolecular Components.” Cell 2016, 165, 1255-1266) reported the performance of a low-cost diagnosis device using a combination of NASBA and the CRISPR/Cas9 technology. This technology is sensitive and robust, but to achieve quantitative measurements, more expensive electrical elements are required. In addition, it takes around 3 hours to perform diagnostics.

### SUMMARY OF THE INVENTION

The disclosure relates to methods for real-time quantification of the amount of a target DNA in a sample, compositions for quantifying the amount of Zika virus in a sample, computer-implemented methods for real-time quantification of the amount of a target DNA in a sample, and a system for real-time quantification of the amount of a target DNA in a sample using loop-mediated isothermal amplification (LAMP).

In accordance with certain embodiments, the method for real-time quantification of the amount of a target DNA in a sample using loop-mediated isothermal amplification (LAMP) is includes the steps of providing a well comprising a sample in a reaction mix. The reaction mix typically includes three pairs of primers that recognize six distinct regions in the target DNA; reverse transcriptase; DNA polymerase; dNTP; and a colorimetric reagent. In this embodiment, the method further includes the steps of heating the well to an amplification temperature between 60° C. to 68° C.; capturing a photo of the well at regular intervals, wherein the regular intervals are one photo every minute after the temperature of the well reaches the amplification temperature; extracting red, green and blue (RGB) values from at least one pixel in the photo of the well; transforming the RGB values from the pixel in the photo of the well into hue, saturation, and value (HSV) values; generating an amplification curve, wherein the amplification curve is a record of changes in an average hue value (h) as a function of time (t) as represented by eq. (1):

$$h = {H}_{ma x} + \frac{{H}_{min} - {H}_{ma x}}{1 + {10}^{S ( {T}_{t} - t )}} , \qquad ( 1 ) ,$$

wherein: Hₘₐₓ is maximum asymptote of the hue value; Hₘᵢₙ is minimum asymptote of the hue value; S is a slope factor; and Tₜ is the time in which h approaches the inflection point; and quantifying the concentration of the target DNA based on the amplification curve. The method may also optionally include the further step of identifying a region of interest (ROI) from which to measure the average h, wherein the region of interest comprises all pixels with a saturation value of at least 0.1.

In yet another embodiment, a composition for quantifying the amount of Zika virus in a sample has at least three pairs of primers that recognize six distinct regions in the ZIKV envelope protein coding region, wherein the first primer of the three pairs of primers comprises a sequence set forth in SEQ ID NO:1, a second primer of the three pairs of primers comprises a sequence set forth in SEQ ID NO:2, a third primer of the three pairs of primers comprises a sequence set forth in SEQ ID NO:3, a fourth primer of the three pairs of primers comprises a sequence set forth in SEQ ID NO:4, a fifth primer of the three pairs of primers comprises a sequence set forth in SEQ ID NO:5, and a sixth primer of the three pairs of primers comprises a sequence set forth in SEQ ID NO:6.

In accordance with certain embodiment, a computer-implemented method for real-time quantification of the amount of a target DNA in a sample using loop-mediated isothermal amplification (LAMP) is provided. In this embodiment, the method typically comprises executing on a processor the steps of: receiving via a computer, photos of a well containing a LAMP reaction, wherein the photos are of the well every one minute during the duration of the LAMP reaction; extracting red, green and blue (RGB) values from at least one pixel in the photo of the well; transforming the RGB values from the pixel in the photo of the well into hue, saturation, and value (HSV) values; creating a data file comprising a record of the HSV values from all of the photos of the well taken during the duration of the LAMP reaction; generating an amplification curve from the data file, wherein the amplification curve is a record of changes in an average hue value (h) as a function of time (t) as represented by eq. (1):

$$h = {H}_{ma x} + \frac{{H}_{min} - {H}_{ma x}}{1 + {10}^{S ( {T}_{t} - t )}} , \qquad ( 1 ) ,$$

wherein: Hₘₐₓ is maximum asymptote of the hue value; Hₘᵢₙ is minimum asymptote of the hue value; S is a slope factor; and Tₜ is the time in which h approaches the inflection point; and quantifying the concentration of the target DNA based on the amplification curve.

In accordance with other embodiment, a system for real-time quantification of the amount of a target DNA is provided comprising: a first memory for storing photos of a well containing a LAMP reaction and the time of the photos were taken, wherein the photos track the LAMP reaction at regular intervals; a processor that transforms the red, green and blue (RGB) values from at least one pixel in a photo of a LAMP reaction into hue, saturation, and value (HSV) values and generates a data file comprising a record of the HSV values from all of the photos of the well taken during the duration of the LAMP reaction; a second memory for storing the data file comprising the record of the HSV values from all of the photos of the well taken during the duration of the LAMP reaction; and a third memory for storing an amplification curve generated by the processor from the data file.

### DESCRIPTION OF THE FIGURES

FIGS. 1A-1D depict one embodiment of the experimental setup for real-time reverse transcription quantitative loop mediated isothermal amplification (RT-qLAMP). FIG. 1A shows the three major components of our RT-qLAMP system: (1) a color camera, (2) a microwell chip with multiple reaction chambers, and (3) a heater that maintains the reaction temperature at a constant 65° C. during amplification. A photograph of the experimental setup is shown in FIG. 1D. FIGS. 1B and 1C show the principle of the colorimetric RT-qLAMP measurement.

FIGS. 2A-2B depict, in accordance with certain embodiments, an overview of the chip at the end point. FIG. 2A shows the results of a sensitivity test of the colorimetric RT-LAMP with (ZIKV RNA template concentration: column 1: 10⁴ copies/μL, column 2: 10³ copies/μL, column 3: 10² copies/μL, column 4: 10 copies/μL, column 5: 0 copies/μL). The five reaction chambers in one column represent five replicates. FIG. 2B shows the results of a primer specificity test with multiple template RNAs (column 1: human FLO-1 cell line RNA, column 2: ZIKV RNA, column 3: DENY RNA, column 4: CHIKV RNA, column 5: WNV RNA).

FIGS. 3A-3F depicts, in accordance with certain embodiments, amplification curves (FIGS. 3A-3E) and standard curve (FIG. 3F) of real-time colorimetric RT-qLAMP of ZIKV RNA.

FIGS. 4A-4E depict, in accordance with certain embodiments, multiparameter fits in relation to DNA concentration. FIG. 4A is directed to percent of wells reacting. FIG. 4B is directed to predicted DNA when the LAMP reaction is performed for longer than the usual reaction period to make the reaction more vulnerable to false positives. FIG. 4C is directed to mean Tₜ in a well. FIG. 4D is directed to the standard deviations for each well. FIG. 4E is directed to predicted DNA for a typical LAMP reaction. FIG. 4F is directed to the relationship among DNA concentration, percent of wells reacting, and mean Tₜ.

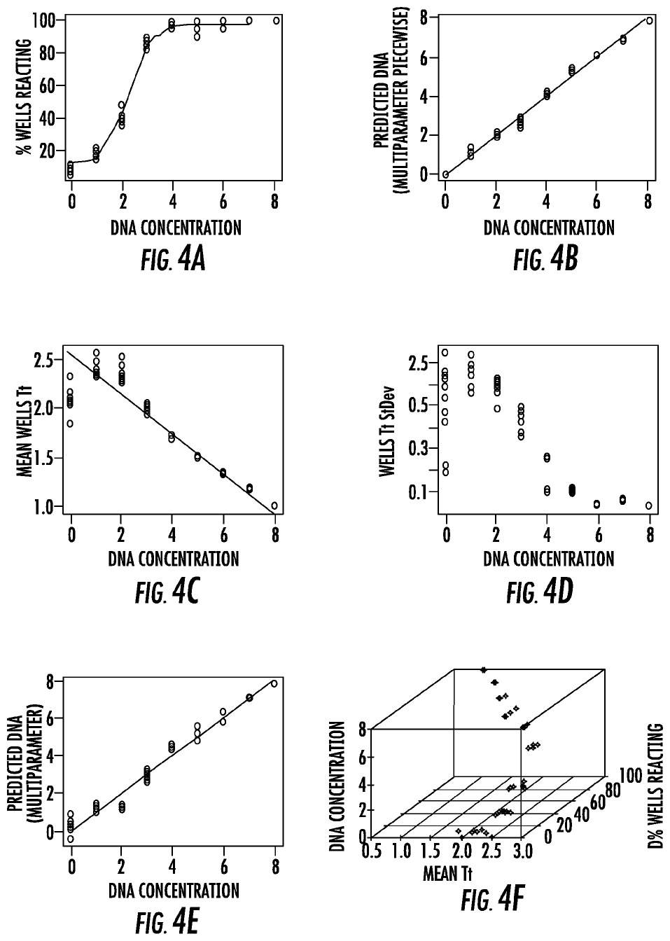

### DETAILED DESCRIPTION

Detailed aspects and applications of the disclosure are described below in the following drawings and detailed description of the technology. Unless specifically noted, it is intended that the words and phrases in the specification and the claims be given their plain, ordinary, and accustomed meaning to those of ordinary skill in the applicable arts.

In the following description, and for the purposes of explanation, numerous specific details are set forth in order to provide a thorough understanding of the various aspects of the disclosure. It will be understood, however, by those skilled in the relevant arts, that embodiments of the technology disclosed herein may be practiced without these specific details. It should be noted that there are many different and alternative configurations, devices and technologies to which the disclosed technologies may be applied. The full scope of the technology disclosed herein is not limited to the examples that are described below.

The singular forms “a,” “an,” and “the” include plural referents unless the context clearly dictates otherwise. Thus, for example, reference to “a step” includes reference to one or more of such steps.

As used herein, the term “about” refers to an range of ±2% or ±2 units from the point of reference. For example, in the context of temperature, a temperature of about 65° C. refers a temperature range of between between 63° C. and 67° C. or between 63.7° C. and 66.3° C. In some embodiments, the term “about” refers to an range of ±1%, ±0.5%, ±0.25%, ±0.1%, ±1 unit, ±0.5 units, ±0.25 units, or ±0.1 unit from the point of reference.

As used herein, the term “computer” refers any of the various form of digital computers, such as laptops, desktops, workstations, personal digital assistants, servers, blade servers, mainframes, and/or other appropriate computers. In some embodiments, a computer comprises a processor, a memory device, and a storage device. In some aspects, the term computer also refers to a mobile computing device, which comprises a mobile compatible processor, a mobile compatible memory, and an input/output device such as a mobile display, for example, a smartphone.

The disclosure presents a simple platform real-time reverse transcription quantitative loop mediated isothermal amplification (RT-qLAMP). In particular, the platform involves LAMP using colorimetric reagents. This is the first demonstration of real-time quantitative and colorimetric viral RNA detection on a chip-level device as well as the first demonstration of real-time colorimetric qLAMP. This system satisfies a long-felt need for economical, rapid, and effective technology suitable for front-line performance for point-of-care diagnostics, especially in the cases of pandemics.

The feasibility of this novel real-time colorimetric RT-qLAMP technology is demonstrated using an inexpensive digital camera and heater. To show that this colorimetric quantification approach is promising for future miniaturization, all analyses were performed on a mass-production compatible, stamp-size plastic microwell chip. The combination of inexpensive components, small volume reaction and robust analysis is particularly suited to resource limited developing countries. When mass produced, the cost of running colorimetric RT-qLAMP detections on the microwell chip can be significantly lower than conventional PCR approach. In addition, the device is small and consumes low energy, and allows for rapid analysis. These features enable point-of-care diagnosis and analysis in remote areas. Professionals, or people without specific knowledge, would be able to make their own microwell chip.

The disclosure is directed to methods of real-time quantification of the concentration of a target DNA in a sample using LAMP. The methods comprise first providing a well (for example, a microwell) comprising a sample (for example, a RNA or DNA sample) in a reaction mix. The reaction mix comprises at least one pair of primer or at least two pairs of primers (for example, three pairs of primers), a reverse transcriptase, a DNA polymerase, dNTP, and a colorimetric reagent. In certain embodiments, each of the primers recognizes distinct regions in the target DNA. The well is then heated to an amplification temperature (for example, between 60° C. to 72° C., between 65° C. to 72° C., between 68° C. to 72° C., between 60° C. to 70° C., between 65° C. to 70° C., between 68° C. to 70° C., between 60° C. to 68° C., between 65° C. to 68° C., between 60° C. to 65° C., or at 65° C.). For the duration of the amplification reaction in the well (for example, starting when the well reaches the amplification temperature), a colored photo of the well is captured at regular intervals, for example, every second, every 5 seconds, every 10 seconds, every 15 seconds, every 30 seconds, every minute, every 2 minutes, every 3 minute, every 4 minutes, or every 10 minutes. In some implementation, the period of photo-capture is at least 30 minutes, at least 35 minutes, or at least 40 minutes. In another implementation, the period of photo-capture is no more than 60 minutes. In some implementations, for example for slower reactions, the interval for capturing the photo of the well is at longer intervals, such as taking the photos at an interval of more than one minutes. When photos are captured at longer intervals, it is important to control lighting to reduce the amount of noise in the data. The quality of the camera will also help reduce the amount of noise in the data.

The red, green and blue (RGB) values from at least one pixel in the photo of the well is calculated and then converted into hue, saturation, and value (HSV) values. An amplification curve, which is a record of changes in an average hue value (h) as a function of time (t), is generated using the formula as represented by eq. (1):

$$h = {H}_{ma x} + \frac{{H}_{min} - {H}_{ma x}}{1 + {10}^{S ( {T}_{t} - t )}} , \qquad ( 1 ) ,$$

wherein Hₘₐₓ is maximum asymptote of the hue value; Hₘᵢₙ is minimum asymptote of the hue value; S is a slope factor; and Tₜ is the time in which h approaches the inflection point.

The disclosure is also directed to methods of electronically capturing LAMP data for real-time quantification of the amount of a target DNA in a sample, for example, computer-implemented methods for real-time quantification of the amount of a target DNA in a sample using LAMP. The methods comprise executing on a processor a plurality of computer-executable instructions (e.g., stored on a non-transitory computer-readable medium), wherein execution of the instructions causes the processor to perform some or all of the following steps: receiving a photo of a well containing a LAMP reaction every one minute during the duration of the LAMP reaction in a computer; extracting RGB values from at least one pixel in the photo of the well; transforming the RGB value from the pixel in the photo of the well into HSV values; creating a data file comprising a record of the HSV values from all of the photos of the well taken during the duration of the LAMP reaction; and generating an amplification curve from the data file, wherein the amplification curve is a record of changes in an average hue value (h) as a function of time (t) as represented by eq. (1):

$$h = {H}_{ma x} + \frac{{H}_{min} - {H}_{ma x}}{1 + {10}^{S ( {T}_{t} - t )}} , \qquad ( 1 ) ,$$

wherein is maximum asymptote of the hue value; Hₘᵢₙ is minimum asymptote of the hue value; S is a slope factor; and Tₜ is the time in which h approaches the inflection point.

In some implementations, the method further comprises identifying a region of interest (ROI) from which to measure the average h. In certain embodiments, the ROI includes all pixels in the colored with a saturation value of at least a cut-off saturation value. This requirement allows for flexible well formation and is beneficial for analyzing sample-containing devices with complicated geometry, such as microfluidic devices or devices that are vulnerable to bubbles. The background outside of the reaction wells and air bubbles reflect ambient illumination and tend to exhibit low saturation, which provide the basis to reject bubbles during image processing. For the computer-implemented methods, the data file comprises average h for each photo of the well calculated from an average of the h in the region of interest.

Depending on the lighting, camera, and the concentration of colorimetric reagent, saturations in different batches of reactions vary. Within each batch of reaction, there is a significant difference between the saturation of the pixels in the reaction region and the background region. The difference in saturation of the reaction and the background region enable the determination of a cut-off value to classify the reaction and background regions. As the saturation values can vary, the cut-off value for the saturation value may be unique to each reaction set up. In some implementations, the cut-off saturation value is determined by randomly sampling the pixels from the reaction regions and background regions, calculating the saturations of each pixel in these regions, choosing a saturation value that differentiate between reaction and background regions, and applying this cut-off saturation value to analyze the whole stack of reaction images. In some implementations, the cut-off saturation value is 0.1.

The methods of the disclosure may further comprise quantifying the concentration of the target DNA in the sample as a function of Tₜ. In some implementations, Tₜ is converted into quantification information using methods for converting the threshold cycle (Ct) value in traditional quantitative PCR into quantification information where Ct is replaced with Tₜ. In certain embodiments, absolute quantification is used to convert Tₜ into an expression level based on a standard curve. Accordingly, a standard curve is generated using known amounts of DNA in the sample, and then the concentration of target DNA is estimated from the standard curve. In other embodiments, relative quantification is used to convert Tₜ into an expression level, in particular via ΔΔTₜ, where 2^{-ΔΔT}ₜ is the normalized expression ratio of the target DNA. For example, the steps of relative quantification comprises normalizing the Tₜ of the target DNA to that of a reference gene to produce a ΔTₜ value; normalizing the ΔTₜ value of the test sample to a calibrator to produce a ΔΔTₜ; and determining the normalized gene expression according to the formula: 2^{-ΔΔT}ₜ. The reference gene is a stably expressed gene under different conditions and be chosen used for normalization of other genes

In a particular implementation, the methods quantify the amount of Zika virus in a sample. The amount of Zika virus may be quantified with primers targeted to conserved regions in the Zika virus genome. For example, the amount of Zika virus is quantified through detecting and quantifying the ZIKV envelope protein coding region, such as position 1279-1497 bp of the ZIKV envelope protein coding region (based on Accession No. NC_012532.1). In one embodiment, where there are three pairs of primers in the reaction, the first primer comprises a sequence set forth in SEQ ID NO:1, a second primer comprises a sequence set forth in SEQ ID NO:2, a third primer comprises a sequence set forth in SEQ ID NO:3, a fourth primer comprises a sequence set forth in SEQ ID NO:4, a fifth primer comprises a sequence set forth in SEQ ID NO:5, and a sixth primer comprises a sequence set forth in SEQ ID NO:6. For example, the three pairs of primers in these methods are the primers listed in Table 1. In other embodiments where there are three pairs of primers in the reaction, the first primer consists of the sequence set forth in SEQ ID NO:1 and an additional one, two, three, four, or five nucleotides; the second primer consists of the sequence set forth in SEQ ID NO:2 and an additional one, two, three, four, or five nucleotides; the third primer consists of the sequence set forth in SEQ ID NO:3 and an additional one, two, three, four, or five nucleotides; the fourth primer consists of the sequence set forth in SEQ ID NO:4 and an additional one, two, three, four, or five nucleotides; the fifth primer consists of the sequence set forth in SEQ ID NO:5 and an additional one, two, three, four, or five nucleotides; and the sixth primer consists of the sequence set forth in SEQ ID NO:6 and an additional one, two, three, four, or five nucleotides.

The disclosure is also directed to compositions for quantifying the amount of Zika virus in a sample, such as using LAMP. The composition comprises at least two pairs of primers that recognize six distinct regions in the ZIKV envelope protein coding region. For example, the composition comprises three pairs of primers, wherein a first primer comprises a sequence set forth in SEQ ID NO:1, a second primer comprises a sequence set forth in SEQ ID NO:2, a third primer comprises a sequence set forth in SEQ ID NO:3, a fourth primer comprises a sequence set forth in SEQ ID NO:4, a fifth primer comprises a sequence set forth in SEQ ID NO:5, and a sixth primer comprises a sequence set forth in SEQ ID NO:6. In one embodiment, the composition comprises a first primer of the three pairs of primers consists of a sequence set forth in SEQ ID NO:1, a second primer of the three pairs of primers consists of a sequence set forth in SEQ ID NO:2, a third primer of the three pairs of primers consists of a sequence set forth in SEQ ID NO:3, a fourth primer of the three pairs of primers consists of a sequence set forth in SEQ ID NO:4, a fifth primer of the three pairs of primers consists of a sequence set forth in SEQ ID NO:5, and a sixth primer of the three pairs of primers consists of a sequence set forth in SEQ ID NO:6. In other embodiments, the composition comprises a first primer consisting of the sequence set forth in SEQ ID NO:1 and an additional one, two, three, four, or five nucleotides; a second primer consisting of the sequence set forth in SEQ ID NO:2 and an additional one, two, three, four, or five nucleotides; a third primer consisting of the sequence set forth in SEQ ID NO:3 and an additional one, two, three, four, or five nucleotides; a fourth primer consisting of the sequence set forth in SEQ ID NO:4 and an additional one, two, three, four, or five nucleotides; a fifth primer consisting of the sequence set forth in SEQ ID NO:5 and an additional one, two, three, four, or five nucleotides; and a sixth primer consisting of the sequence set forth in SEQ ID NO:6 and an additional one, two, three, four, or five nucleotides.

### Illustrative, Non-Limiting Example in Accordance with Certain Embodiments

The disclosure is further illustrated by the following examples that should not be construed as limiting. The contents of all references, patents, and published patent applications cited throughout this application, as well as the Figures, are incorporated herein by reference in their entirety for all purposes.

### Colorimetric RT-qLAMP

The basic approach to the colorimetric RT-qLAMP analysis relies on the color change that occurs as the amplicon accumulates during an RT-qLAMP reaction. Our experimental setup tracks the colors of each RT-qLAMP reaction over time to follow reaction progression. All reaction chambers are monitored over time. As reactions proceed, the colors of positive reactions shift from purple to sky blue. The time period of the color change during amplification may be used to infer the concentration of the target template in the sample in a manner similar to the determination of the threshold cycle ‘Ct’ value of qPCR.

1. Reaction Mixture

The RT-qLAMP primers designed for targeting the ZIKV RNA target the conserved regions on ZIKV genome to distinguish ZIKV from other flaviviruses. These conserved regions include envelope protein coding region, NS5 coding sequence, and NS3 coding sequence. In order to test the sensitivity, quantification capability, and specificity of the RT-qLAMP reaction, six primers targeting the ZIKV envelope protein coding region (position 1279-1497 bp) were designed. The primers were analyzed and optimized in silico to reduce the formation of secondary structures, including self-dimers and cross-dimers. The targeted sequence of the primers has been compared with the genomes of other common flavivirus to ensure that the target sequence shared high homology in ZIKV and low identity in other viruses. The primer sequences are listed in Table 1.

TABLE 1
RT-qLAMP primer sequences and concentrations

| Primer | | | Concentration |
|---|---|---|---|
| name | Sequence (5′ to 3′) | SEQ ID NO: | (μM) |
| FTP | CCGGTTGAATGCTCTTCCCGGGCAAAGGGAGCTTGGTGAC | 1 | 1.6 |
| BIP | GCTATCAGTGCATGGCTCCCAGGCGTAACCTCGACTTTCG | 2 | 1.6 |
| LF | AACACGTAAACTTGGCACAT | 3 | 0.8 |
| LB | AGCGGGATGATTGGATATGAA | 4 | 0.8 |
| F3 | GGGAAACGGTTGTGGACTT | 5 | 0.2 |
| B3 | GCTTCCGCTCTTGGTGAAT | 6 | 0.2 |

The RT-qLAMP amplification procedure was modified based on the previous work described by Tomita et al. (“Loop-mediated isothermal amplification (LAMP) of gene sequences and simple visual detection of products.” Nat. Protoc. 2008, 3, 877-882) and Goto et al. (“Colorimetric detection of loop-mediated isothermal amplification reaction by using hydroxy naphthol blue.” BioTechniques 2009, 46, 167-172). The RT-qLAMP reaction was performed using 300 units/mL Warmstart RTx reverse transcriptase and 800 units/mL Bst 2.0 Warmstart DNA polymerase in 1× ThermolPol DF buffer (New England Biolabs, Ipswich, Mass.). The reaction solution also contains primer mix (Integrated DNA Technologies, Coralville, Iowa) with concentrations indicated in Table 1, 180 μM HNB (Sigma-Aldrich, St. Louis, Mo.), 6 mM MgSO₄, and 800 μM dNTP. A serial 10-fold dilution of viral RNA was added as reaction templates. In negative control, ddH2O was used to replace the viral RNA.

For proof-of-concept, the performance of the colorimetric quantification method was evaluated using ZIKV genomic RNA (VR-1838DQ™, ATCC, Manassas, Va.). This RNA was obtained from the ZIKV strain MR 766. The specificity of the Zika RT-qLAMP primers was tested by human cell line FLO-1 RNA, dengue virus (DENV) RNA (ATCC® VR-3228SD™ ATCC, Manassas, Va.), chikungunya (CHIKV) RNA (ATCC® VR-3246SD™, ATCC, Manassas, Va.), and West Nile Virus (WNV) RNA (ATCC® VR-3198SD™, ATCC, Manassas, Va.).

Real-time colorimetric quantification on an acrylic microwell chip that contained a 5×5 array of microwells. The volume of each microwell is 4 μL. The microwell chip was rinsed by 0.1% micro90 (Sigma-Aldrich, St. Louis, Mo.), followed by ddH₂O, to remove potential contaminants. The microwell chip for the study was made with acrylic, but fabrication, but other types of plastics, for example, cyclic olefin polymers, polypropylene, polystyrene, could be suitable for colorimetric LAMP reactions as well (data not published). Instead of laser-cutting, the reaction chambers could be produced by other existing plastic forming methods.

2. Colorimetric Detection

To represent color changes during RT-qLAMP, it is desired that a color space is applied in which any color is a single value that is independent of brightness and illumination. The computer converts the color of the digital image from the RGB (Red, Green, Blue) space into the HSV (Hue, Saturation, Value) space. HSV35 is a color space for describing color properties. It uses a hexone model in the H, S, and V axes in a cylindrical coordinate system to represent colors. The hue value is the azimuth, defined from red at 0°, yellow at 60°, green at 120°, blue at 240°, and back to red again at 360° (0°). The hue value can be used as a single quantity for tracking color changes as RT-qLAMP reactions proceed.

Colorimetric detection starts by estimating the radius of a reaction chamber on real-time images. Based on the radius of the reaction chamber shown on the image, square Region of Interest (ROI), which can be fitted into the reaction chamber area, is defined. To reduce the variations, the same side length is applied to all of the ROIs in one experiment. Once an ROI of a specific reaction chamber was defined, its location and area were not changed until the whole stack of images was processed. The RGB (Red, Green, Blue) values of all of the pixels in each ROI was extracted. The RGB values from each pixel were transformed into HSV color space. The HSV values of each ROI were obtained by calculating the mean H, S, and V of all the pixels that passed the bubble and background rejection algorithm. The mean H, S, and V of each ROI were used to represent the color information of the corresponding reaction chamber.

3. Region of Interest (ROI) Identification

The goal of the ROI identification algorithm is to automatically exclude the undesired pixels in the image that do not contain LAMP mixture during colorimetric detection. The algorithm identified the locations and size of the reaction wells, and automatically rejected the foreign objects or bubbles. The algorithm allows for flexible microwells formation, and is beneficial to devices with complicated geometry such as microfluidic devices or devices that are vulnerable to bubbles. The background outside of the reaction microwells and air bubbles reflected ambient illumination and tended to exhibit low saturation, which provided the basis to reject bubbles during image processing. Saturation S is the radial coordinate in HSV color space that shows the purity of a color. For this study, threshold on the saturation was set as S=0.1 When the S of a pixel was below the threshold, the algorithm rejected the pixel. All pixels that passed the bubble and background rejection algorithm were used to compute the average hue.

3. Curve-Fitting Algorithm

The record of hue changes as a function of time represents the RT-qLAMP amplification curve. Similar to qPCR, this amplification reaction is best characterized as a sigmoid function. Real time hue data is fit based on the sigmoid function shown in Eq. (1):

$$h = {H}_{ma x} + \frac{{H}_{min} - {H}_{ma x}}{1 + {10}^{S ( {T}_{t} - t )}} , \qquad ( 1 )$$

where h is the hue value of the reaction as a function of reaction time t. Hmax and Hmin are the maximum and minimum asymptotes, respectively. Similar to the threshold cycle (Ct) value in qPCR, the threshold time of the reaction (Tₜ) represents the time when the hue value approaches the inflection point. Tₜ is also the time when the derivative of the curve displays a maximal absolute value and the time the amplification has the highest rate. S is a slope factor. Four parameters, Hₘₐₓ, Hₘᵢₙ, Tₜ, and S, were estimated by nonlinear least-squares.

4. Experimental Protocol and Results

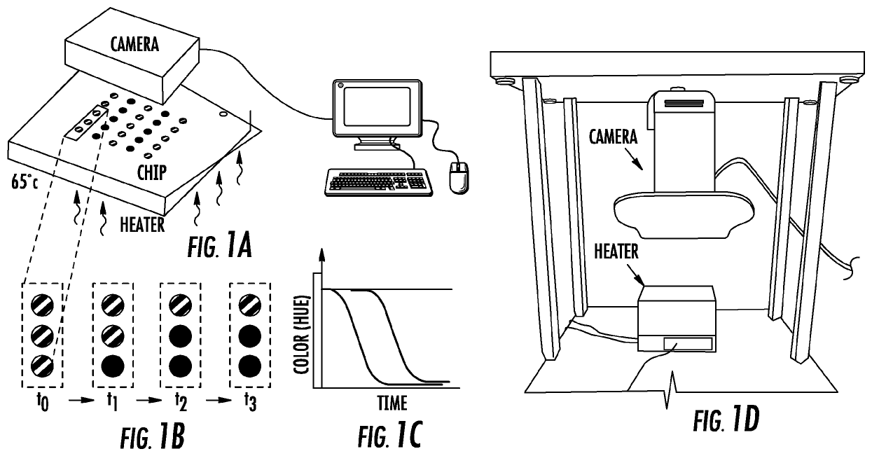

The RT-qLAMP reaction solution was prepared using the aforementioned protocol. Silicone oil (Fisher Scientific, Pittsburgh, Pa.) was loaded into the reaction chambers before the reaction solution to prevent cross contamination during the loading process. Reaction solutions of 3 μL were loaded into each reaction chamber in the microwell chip. The chip was placed in a custom-made heater (FIG. 1 Panel D) to maintain a constant reaction temperature of 65° C. for one hour. A webcam (HD Pro Webcam C920, Logitech, Newark, Calif.) was placed above the chip to monitor the reaction. Color images were captured and recorded every one minute. In this experiment, the color is easily visible and captured by cameras under regular room lighting and white background. The color shift of the reaction was characterized by real-time hue change. Following the image analysis pipeline, the real-time raw hue data was fitted by the sigmoid function. The parameter Tₜ from the sigmoid function shown in Eq. (1) was used to represent the reaction rate.

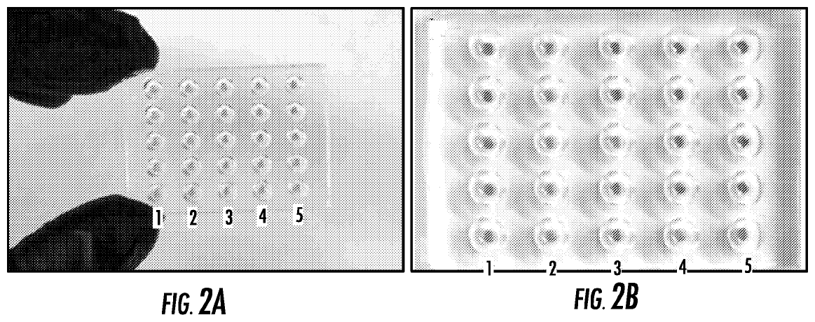

FIG. 2 shows an overview of the microwell chip with colorimetric RT-qLAMP reactions at the end point. Five groups of reactions were performed simultaneously on the microwell chip. For each group, five replicate experiments were performed in five isolated reaction chambers. FIG. 2 Panel A is an end point image demonstrating the detection limit of the colorimetric RT-qLAMP reaction. All reactions with template ZIKV RNA concentration higher than 10 copies/μL turned to sky blue at the end of the reaction, while all of the negative controls were purple. Four out of five reactions turned to positive in the 10 copies/μL group, indicating that the detection limit was close to single copies of target ZIKV RNA. In clinical samples, the ZIKV RNA loads have been reported to be 2.2×10⁸ copies/mL in urine, 2.1×10⁶ copies/mL in breast milk, and 7.3×10⁵ copies/mL in serum. This suggested that the colorimetric RT-qLAMP technology should be sensitive enough to detect the ZIKV RNA from clinical samples. The specificity of the ZIKV primers was tested (FIG. 2 Panel B). No cross reactivity was observed between the ZIKV primers and the non-target RNAs. Only the positive control with ZIKV RNA showed sky blue, while the reactions with human cell line RNA, DENV RNA, CHIKV RNA, and WNV RNA remained purple. This result confirmed that the ZIKV primers were specific to Zika viral RNA.

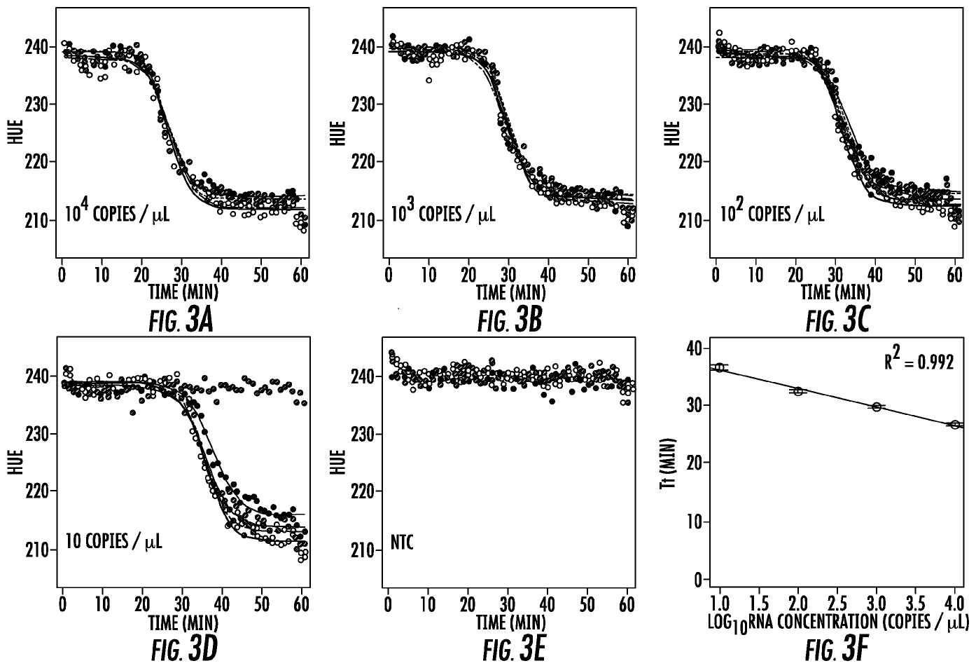

Proof of real-time quantitation using colorimetric RT-qLAMP is shown in FIG. 3. Panels A-E show amplification curves of how hue shifts in negative and positive RT-qLAMP reactions. The data was taken from the same experiment as the one shown in FIG. 2 Panel A. The real-time raw hue values were plotted as dots and the values were fitted by the sigmoid function (Eq. 1) using solid curves. Each color represents a replicate within the same template concentration group. Hue values decreased from around 240 to around 210 in most positive RT-qLAMP reactions (from 10 copies/μL to 104 copies/μL), but remained constant at around 240 in negative controls.

The Tₜ values of positive RT-qLAMP reactions have been calculated and plotted in FIG. 3 Panel F. In this experiment, the color shift of all positive reactions was achieved within 40 minutes. There is a strong linear trend between the Tₜ values and log₁₀ RNA concentrations. As RT-qLAMP proceeds rapidly, a 10-fold difference in template RNA results in an approximately 3-minute ΔTₜ in this specific experiment. Even though this ΔTₜ is small, the error bars on the standard curve were also small, suggesting that there were small variations among the Tₜ values with the same template RNA concentration. Using this standard curve, one can easily tell the template RNA concentration in a reaction based on a Tₜ value. This result indicated that real time colorimetric RT-qLAMP can be a promising candidate for characterizing the viral RNA loads in routine practice.

The colorimetric detection approach reported could be completed within 40 minutes. The results show that this technology can specifically identify ZIKV RNA, showing no cross-reactivity with other common viral RNAs. These characteristics suggest the real-time colorimetric ZIKV RNA detection platform was suitable for both rapid point-of-care diagnostics and front-line Zika monitoring. The highly quantitative feature of this technology indicates the platform can be used in rapid viral load tests for vaccine and medication development purposes. The chemical reaction, colorimetric RT-qLAMP, is robust to multiple materials used in reaction vessels. The protocol is portable to different lab ware formats, from tubes, microplates, and microwell chips (as presented in this work) to future microfluidic cassettes.

Most of the current Zika detection technologies must be performed in a laboratory setting and require a long period of time for detection. They are limited for field operations in resource-limited areas. In our real-time colorimetric ZIKV RNA quantification technology, the amplification can be monitored by a ubiquitous color camera or webcam. In one scenario, health workers can perform the RT-qLAMP on a heating plate or in an oven at 65° C. and monitor the color changes using a cell phone. The results can be analyzed on the phone or on a cloud server. This approach does not require new technology or infrastructure, but allows rapid assessment of viral outbreaks from personal to societal levels.

1. A method for real-time quantification of the amount of a target DNA in a sample using loop-mediated isothermal amplification (LAMP), the method comprising:
providing a well comprising a sample in a reaction mix, the reaction mix comprising:
three pairs of primers, wherein the three pairs of primers recognize six distinct regions in the target DNA;
reverse transcriptase;
DNA polymerase;
dNTP; and
a colorimetric reagent;
heating the well to an amplification temperature, wherein the amplification temperature is between 60° C. to 68° C.;
capturing a photo of the well at regular intervals, wherein the regular intervals are one photo every minute after the temperature of the well reaches the amplification temperature;
extracting red, green and blue (RGB) values from at least one pixel in the photo of the well;
transforming the RGB values from the pixel in the photo of the well into hue, saturation, and value (HSV) values;
generating an amplification curve, wherein the amplification curve is a record of changes in an average hue value (h) as a function of time (t) as represented by eq. (1):
$$h = {H}_{ma x} + \frac{{H}_{min} - {H}_{ma x}}{1 + {10}^{S ( {T}_{t} - t )}} , \qquad ( 1 ) ,$$
wherein:
Hₘₐₓ is maximum asymptote of the hue value;
Hₘᵢₙ is minimum asymptote of the hue value;
S is a slope factor; and
Tₜ is the time in which h approaches the inflection point; and
quantifying the concentration of the target DNA based on the amplification curve.

2. The method of claim 1, wherein the well is a microwell.

3. The method of claim 1, wherein the amplification temperature is 65° C.

4. The method of claim 1, wherein the temperature of the well is at the amplification temperature for at least 40 minutes.

5. The method of claim 1, wherein the temperature of the well is at the amplification temperature for no more than 60 minutes.

6. The method of claim 1, further comprising identifying a region of interest (ROI) from which to measure the average h, wherein the region of interest comprises all pixels with a saturation value of at least 0.1.

7. The method of claim 1, wherein the concentration of the target DNA in the sample is quantified as a function of Tₜ.

8. The method of claim 7, wherein the step of quantifying the concentration of the target DNA in the sample as a function of Tₜ comprises substituting the threshold cycle (Ct) value in quantitative PCR with Tₜ.

9. The method of claim 1, wherein the method quantifies the amount of Zika virus in a sample, the target DNA is the ZIKV envelope protein coding region.

10. The method of claim 9, wherein the target DNA is position 1279-1497 bp of the ZIKV envelope protein coding region.

11. The method of claim 10, wherein the first primer of the three pairs of primers comprises a sequence set forth in SEQ ID NO:1, a second primer of the three pairs of primers comprises a sequence set forth in SEQ ID NO:2, a third primer of the three pairs of primers comprises a sequence set forth in SEQ ID NO:3, a fourth primer of the three pairs of primers comprises a sequence set forth in SEQ ID NO:4, a fifth primer of the three pairs of primers comprises a sequence set forth in SEQ ID NO:5, and a sixth primer of the three pairs of primers comprises a sequence set forth in SEQ ID NO:6.

12. The method of claim 10, wherein the first primer of the three pairs of primers consists of a sequence set forth in SEQ ID NO:1, a second primer of the three pairs of primers consists of a sequence set forth in SEQ ID NO:2, a third primer of the three pairs of primers consists of a sequence set forth in SEQ ID NO:3, a fourth primer of the three pairs of primers consists of a sequence set forth in SEQ ID NO:4, a fifth primer of the three pairs of primers consists of a sequence set forth in SEQ ID NO:5, and a sixth primer of the three pairs of primers consists of a sequence set forth in SEQ ID NO:6.

13. The method of claim 1, wherein the colorimetric reagent is HNB.

14. A composition for quantifying the amount of Zika virus in a sample, the composition comprising three pairs of primers that recognize six distinct regions in the ZIKV envelope protein coding region, wherein the first primer of the three pairs of primers comprises a sequence set forth in SEQ ID NO:1, a second primer of the three pairs of primers comprises a sequence set forth in SEQ ID NO:2, a third primer of the three pairs of primers comprises a sequence set forth in SEQ ID NO:3, a fourth primer of the three pairs of primers comprises a sequence set forth in SEQ ID NO:4, a fifth primer of the three pairs of primers comprises a sequence set forth in SEQ ID NO:5, and a sixth primer of the three pairs of primers comprises a sequence set forth in SEQ ID NO:6.

15. The composition of claim 14, wherein the first primer of the three pairs of primers consists of a sequence set forth in SEQ ID NO:1, a second primer of the three pairs of primers consists of a sequence set forth in SEQ ID NO:2, a third primer of the three pairs of primers consists of a sequence set forth in SEQ ID NO:3, a fourth primer of the three pairs of primers consists of a sequence set forth in SEQ ID NO:4, a fifth primer of the three pairs of primers consists of a sequence set forth in SEQ ID NO:5, and a sixth primer of the three pairs of primers consists of a sequence set forth in SEQ ID NO:6.

16-21. (canceled)

22. A system for real-time quantification of the amount of a target DNA in a sample using loop-mediated isothermal amplification (LAMP), the system comprising:
a first memory for storing photos of a well containing a LAMP reaction and the time of when the photos were taken, wherein the photos track the LAMP reaction at regular intervals;
a processor that transforms the red, green and blue (RGB) values from at least one pixel in a photo of a LAMP reaction into hue, saturation, and value (HSV) values and generates a data file comprising a record of the HSV values from all of the photos of the well taken during the duration of the LAMP reaction;
a second memory for storing the data file comprising the record of the HSV values from all of the photos of the well taken during the duration of the LAMP reaction in relation to time of when the photos were taken; and
a third memory for storing an amplification curve generated by the processor from the data file, wherein the amplification curve is a record of changes in an average hue value (h) as a function of time (t) as represented by eq. (1):
$$h = {H}_{ma x} + \frac{{H}_{min} - {H}_{ma x}}{1 + {10}^{S ( {T}_{t} - t )}} , \qquad ( 1 ) ,$$
wherein:
Hₘₐₓ is maximum asymptote of the hue value;
Hₘᵢₙ is minimum asymptote of the hue value;
S is a slope factor; and
Tₜ is the time in which h approaches the inflection point.

23. The system of claim 22, furthering comprising a camera, wherein the processor instructs the camera to take photos of a well containing a LAMP reaction at regular intervals during the duration of the LAMP reaction.

24. The system of claim 23, wherein the photos are taken every minute.

25. The system of claim 22, wherein the processor transforms the RGB values from at least one pixel in a region of interest in the photo of a LAMP reaction into HSV values, wherein the region of interest excludes pixels in the photo that do not contain LAMP mixture.

26. The system of claim 22, wherein the processor transforms the RGB values from at least one pixel in a region of interest in the photo of a LAMP reaction into HSV values, wherein the region of interest comprises all pixels with a saturation value of at least 0.1.
